(19) United States
(12) Patent Application Publication
(10) Pub. No.: US 2019/0015395 A1
(43) Pub. Date: Jan. 17, 2019

(54) TACROLIMUS FOR TREATING TDP-43 PROTEINOPATHY

(71) Applicant: CHRONOS THERAPEUTICS LIMITED, Oxford (GB)

(72) Inventors: Pete APPLEFORD, Oxford (GB); Huw JONES, Guilden Morden (GB); Helen KUHLMAN, Bath (GB); Julia SHERRIFF, Oxford (GB)

(73) Assignee: Chronos Therapeutics Limited, Oxford (GB)

(21) Appl. No.: 16/068,558

(22) PCT Filed: Jan. 6, 2017
(86) PCT No.: PCT/GB2017/050021

§ 371 Date: Jul. 6, 2018

(30) Foreign Application Priority Data
Jan. 8, 2016 (GB) 1600376.3

(51) Int. Cl. A61K 31/436; A61P 25/00

(52) U.S. Cl. CPC A61K 31/436; A61P 25/00

(57) ABSTRACT

The present invention provides tacrolimus for use in the treatment in a human subject of a TDP-43 proteinopathy, such as amyotrophic lateral sclerosis (ALS) and frontotemporal dementias (FTDs), wherein the tacrolimus is for administration at a therapeutically-effective dose which does not cause immunosuppression in the subject.

### FIELD OF INVENTION

The present invention relates to the use of tacrolimus, at low doses, to treat neurological diseases and disorders associated with the formation of TDP-43 aggregates within the central nervous system.

### BACKGROUND

Amyotrophic lateral sclerosis (ALS), sometimes called Lou Gehrig's disease, is a rapidly progressive, invariably fatal neurological disease that attacks the neurons responsible for controlling voluntary muscles (such as those in the arms, legs, and face). The disease belongs to a group of disorders known as motor neuron diseases, which are characterised by the gradual degeneration and death of motor neurons.

Signals from motor neurons in the brain (called upper motor neurons) are transmitted to motor neurons in the spinal cord (called lower motor neurons) and then onward from there to specific muscles. In ALS, both the upper motor neurons and the lower motor neurons degenerate or die, leading to a loss of ability to stimulate muscles. Unable to function, the muscles gradually weaken, waste away (atrophy), and have very fine twitches (called fasciculations). Eventually, the ability of the brain to start and control voluntary movement is lost.

ALS causes weakness with a wide range of disabilities. Eventually, all muscles under voluntary control are affected, and individuals lose their strength and the ability to move their arms, legs, and body. When muscles in the diaphragm and chest wall fail, people lose the ability to breathe without ventilatory support. Most people with ALS die from respiratory failure, usually within 3 to 5 years from the onset of symptoms. However, about 10 percent of those with ALS survive for 10 or more years.

ALS is one of the most common neuromuscular diseases worldwide, and people of all races and ethnic backgrounds are affected. ALS has an average global prevalence of 2-7 per 100,000, higher in the UK and USA than many other countries (estimates for the UK are ca. 5,000 ALS patients implying prevalence of over 8 per 100,000). Apart from the clear detrimental effect ALS has on the individual affected and their family, ALS also has an economic impact. These costs can be divided into three components: direct costs, indirect costs and intangible costs. In 2010 the Lewin Group estimated the economic impact of ALS in the US to be $1.03 billion per annum using a moderate prevalence model. The per-patient cost per annum was estimated to be $63,848 and end of life care costs approx. $200,000.

In 90-95% of all ALS cases, the disease occurs apparently at random with no clearly associated risk factors. Individuals with this sporadic form of the disease do not have a family history of ALS, and their family members are not considered to be at increased risk for developing it.

In contrast, about 5-10% of all ALS cases are inherited. This familial form of ALS usually results from a pattern of inheritance that requires only one parent to carry the gene responsible for the disease. Mutations in more than a dozen genes have been found to cause familial ALS.

Despite this diverse etiology of the disease, 97% of patients with ALS display a common phenotype in disease-affected tissues, namely the deposition of the TAR-DNA binding protein (TDP)-43. Deposition of TDP-43 is also the major feature of certain frontotemporal dementias (FTD), associated frontotemporal lobar degeneration (FTLD), which show clinical overlap with ALS.

The role of TDP-43 in ALS is discussed in detail in Scotter et al., 2015, Neurotherapeutics 12(2): 352-363 (the disclosures of which are incorporated herein by reference).

Expression and Function of TDP-43

TDP-43, encoded by TARDBP, is a ubiquitously expressed DNA-/RNA-binding protein. TDP-43 contains two RNA recognition motifs, a nuclear localisation sequence (NLS), a nuclear export signal, and a glycine-rich C-terminus that mediates protein-protein interactions. TDP-43 predominantly resides in the nucleus, but is capable of nucleocytoplasmic shuttling. In the nucleus, TDP-43 plays a critical role in regulating RNA splicing, as well as modulating microRNA biogenesis. TDP-43 can regulate the stability of its own mRNA, providing a mechanism for the autoregulation of TDP-43 protein levels. In addition to TDP-43 RNA, TDP-43 regulates the splicing and stability of a large number of other transcripts, and thus influences diverse cellular processes.

Although mostly nuclear, up to ˜30% of TDP-43 protein can be found in the cytoplasm, with nuclear efflux regulated by both activity and stress. TDP-43 is a key component of dendritic and somatodendritic RNA transport granules in neurons, and plays an important role in neuronal plasticity by regulating local protein synthesis in dendrites. TDP-43 is also involved in the cytoplasmic stress granule response—the formation of protein complexes that sequester mRNAs redundant for survival—meaning TDP-43 function is particularly important under conditions of cellular stress.

TDP-43 Proteinopathy

TDP-43 protein was identified as a major component of the ubiquitinated neuronal cytoplasmic inclusions deposited in cortical neurons in FTD and in spinal motor neurons in ALS. TDP-43-positive inclusions have subsequently been shown to be common to 97% of ALS cases, whether sporadic or familial. The main exceptions are cases caused by mutations in SOD1 or FUS. Neurodegenerative diseases linked to the deposition of TDP-43 are termed “TDP-43 proteinopathies”, and “TDP-43 proteinopathy” also describes the characteristic histopathological transformation of TDP-43 that occurs in disease. This transformation is evidenced by the deposition of full-length and fragmented TDP-43 protein as detergent-resistant, ubiquitinated and hyperphosphorylated aggregates in the cytoplasm, with associated clearing of TDP-43 from the nucleus. The regional spread of TDP-43 proteinopathy from spinal and cortical motor neurons and glia to other cortical regions can be used to stage ALS progression, indicative of some or all of the features of transformed TDP-43 protein being linked to pathogenesis.

A number of studies have examined the roles of TDP-43 gain or loss of function in disease. Overexpression of wild-type TDP-43 recapitulates TDP-43 proteinopathy and disease phenotypes in a range of animal models, supporting a role for gain of toxic function in disease. Initial studies testing a loss-of-function hypothesis used knock-out of TDP-43 from mice, which resulted in embryonic lethality. This demonstrated TDP-43 to play a vital role in early development without necessarily demonstrating that loss of function could be degenerative in adulthood. However, conditional and partial knockout models soon demonstrated that loss of TDP-43 function can, indeed, induce motor neuron defects, a progressive motor phenotype reminiscent of human disease, and even typical TDP-43 proteinopathy. Moreover, either overexpression or knockdown of TDP-43 selectively in glia or muscle also recapitulates ALS-like phenotypes. Thus, it would appear that both gain and loss of TDP-43 function may be mechanistic in disease, and TDP-43 misfolding may link the two.

The Challenge of Developing Therapeutic Agents for ALS

The development of therapeutically effective treatments for ALS has proven to present a considerable challenge to the pharmaceutical industry (see Perrin, 2014, Nature 507:423-425, the disclosures of which are incorporated herein by reference).

Over the last decade, about a dozen different experimental treatments have entered clinical trials in patients with ALS. All had previously been shown to ameliorate symptoms or markers of the disease in an established animal model. However, all but one of these experimental treatments failed to show a therapeutic benefit in humans, and the survival benefits in that one (riluzole) are marginal.

Riluzole (Rilutek®, Sanofi) is currently the only approved treatment for ALS; it is a neuroprotective drug that blocks glutamatergic neurotransmission in the central nervous system, thereby preventing apoptosis (programmed cell death) of the motor neuron.

Accordingly, there is a need for improved therapies for the treatment of TDP-43 proteinopathies, such as ALS. The development of efficacious therapies will serve not only to improve quality and longevity of life for those with the disease but will also aid in lowering the cost burden of such diseases.

### SUMMARY OF INVENTION

The first aspect of the invention provides tacrolimus for use in the treatment of a TDP-43 proteinopathy in a human subject. Advantageously, the tacrolimus is for administration at a therapeutically-effective dose which does not cause immunosuppression in the subject.

Tacrolimus (also called fujimycin or FK506) is clinically employed as an immunosuppressant, for example, in patients who have had organ transplants, and for the treatment of ulcerative colitis or certain skin conditions. Commercially available dosage forms of tacrolimus include capsules containing 0.5 mg, 1 mg, 3 mg and 5 mg and ointments for skin conditions where the concentration is 0.05% to 0.19%. Tacrolimus is most commonly administered twice a day for immunosuppression to prevent rejection of transplanted tissues. The clinically employed dose is generally adjusted to produce a whole blood trough concentration of at least 4 ng/mL when seeking to prevent rejection. This is achieved by employing a recommended initial oral dose (two divided doses every 12 hours) which is in the range 0.075 mg/kg/day to 0.2 mg/kg/day which for an average 70 kg patient required two daily doses of about 2.5 mg to 14 mg.

Tacrolimus is available under trade names such as Prograf®, Advagraf® and Protopic®. Other suitable formulations of tacrolimus are described below in relation to the second aspect of the invention.

The chemical structure of tacrolimus, shown overleaf in Formula I, is a macrolide lactone. It was first discovered in 1987 from the fermentation broth of a Japanese soil sample that contained the bacterium Streptomyces tsukubaensis.

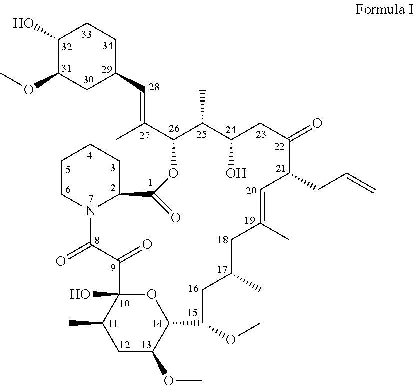 [SMILES: C=CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC]

By “tacrolimus” we include not only the above structure but also related epimers/isomers and other close structural analogues and derivatives that retain the therapeutic efficacy of the above compound in the treatment of TDP-43 proteinopathies.

Suitable epimers/isomers retain the therapeutic efficacy of tacrolimus and may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively, the desired optical isomers may be made by reaction of the appropriate optically active starting materials under conditions which will not cause racemisation or epimerisation, or by derivatisation, for example with a homochiral acid followed by separation of the diastereomeric esters by conventional means (e.g. HPLC, chromatography over silica). All stereoisomers are included within the scope of the invention.

Suitable analogues retain the structure of tacrolimus from C₁ to C₁₇, C₁₉, C₂₀ and C₂₂ to C₃₄ but permit modifications at C₁₈ and/or C₂₁. Such modifications include permitting one hydrogen atom on either of C₁₈ or C₂₁, to be substituted by a C₁₄ alkyl, C₂₄ alkenyl, hydroxyl or methyoxyl group and also include replacing the C₂₁ propenyl group by a hydrogen atom, C₁₋₆ alkyl group, other C₂₋₆ alkenyl group, a hydroxyl group or a methoxyl group. Certain apt compounds include analogues of tacrolimus where the C₂₁ position is modified, for example C₂₁ propenyl group is replaced by a methyl group, an ethyl group or a propyl group. Other apt compounds include analogues where a C₁₈ hydrogen atom is replaced by a hydroxyl group. Certain of these compounds are less immunosuppressant than tacrolimus, for example the analogue wherein the C₂₁ propenyl group is replaced by a methyl group or the analogue wherein a C₁₈ hydrogen is replaced by a hydroxyl group (for example wherein the C₂₁ propenyl group is unchanged or replaced by a methyl, ethyl or propyl group). U.S. Pat. No. 5,376,663 discloses a process for the preparation of analogues of tacrolimus. The C₂₁ propenyl group of tacrolimus may also be replaced by a fluoro C₁₋₆ alkyl group such as a 2-fluoroethyl group or by a different propenyl group such as a 1-methyl ethenyl group.

Thus, in certain embodiments, the active therapeutic agent may be ascomysin (the C21-ethyl analogue of tacrolimus) or dihydrotacrolimus (the C21-propyl analogue of tacrolimus).

Suitable derivatives of tacrolimus may include active metabolites, such as the 31-O-demethyl derivative and the 13-O-demethyl derivative of tacrolimus (see Iwasaki, 2007, Drug Metab. Pharmacokinet. 22(5):328-335 and Barbarino et al., 2013, Pharmacogenet. Genomics 23(10):563-85, and references cited therein; the disclosures of which are incorporated herein by reference).

It will be appreciated by persons skilled in the art that the tacrolimus compound may be counterbalanced by counter-anions. Exemplary counter-anions include, but are not limited to, halides (e.g. fluoride, chloride and bromide), sulfates (e.g. decylsulfate), nitrates, perchlorates, sulfonates (e.g. methane sulfonate) and trifluoroacetate. Other suitable counter-anions will be well known to persons skilled in the art. Thus, pharmaceutically-acceptable derivatives of the compounds of formula I, such as salts and solvates, are also included within the scope of the invention. Salts which may be mentioned include: acid addition salts, for example, salts formed with inorganic acids such as hydrochloric, hydrobromic, sulfuric and phosphoric acid, with carboxylic acids or with organo-sulfonic acids; base addition salts; metal salts formed with bases, for example, the sodium and potassium salts. Suitable solvates include hydrates and alcoholates.

It will be further appreciated by skilled persons that the tacrolimus compound may exhibit tautomerism. All tautomeric forms and mixtures thereof are included within the scope of the invention.

The tacrolimus compound will generally be administered in admixture with a suitable pharmaceutical excipient diluent or carrier selected with regard to the intended route of administration and standard pharmaceutical practice (for example, see Remington: The Science and Practice of Pharmacy, 19^{th} edition, 1995, Ed. Alfonso Gennaro, Mack Publishing Company, Pennsylvania, USA). Suitable routes of administration are discussed below, and include intravenous, ICV and enteral (e.g. oral, gastric and rectal) administration.

The present invention stems from the finding by the inventors that unusually low doses of tacrolimus are capable of producing a therapeutic benefit in a new model of TDP-43 proteinopathy (see Examples below and Mitchell et al., 2015, Acta Neuropathol. Comm. 3:36, the disclosures of which are incorporated herein by reference).

The term “TDP-43 proteinopathy” is used herein to describe neurodegenerative diseases linked to the deposition of TDP-43, including but not limited to amyotrophic lateral sclerosis (ALS), frontotemporal dementias (such as FTD-TDP-43 and FTD-tau), other dementias (such as Alzheimer's disease and Lewy-body dementia), parkinsonism (including Parkinson's disease, Perry syndrome and ALS Parkinsonism-dementia complex of Guam), polyglutamine diseases (such as Huntington's disease) and myopathies (such as sporadic inclusion body myositis), wherein in each case at least a sub-population of patients exhibit TDP-43 pathology. Further details of TDP-43 proteinopathies are described in Gendron et al., 2010, Neuropathol. Appl. Neurobiol. 36:97-112 and Lagier-Tourenne et al., 2010, Hum. Mol. Gen. 19(1):R46-R64; the disclosures of which are incorporated herein by reference.

Thus, in one embodiment, the TDP-43 proteinopathy is amyotrophic lateral sclerosis (ALS). It will be appreciated by persons skilled in the art that the subject to be treated may have familial amyotrophic lateral sclerosis (fALS) or sporadic amyotrophic lateral sclerosis (sALS).

In an alternative embodiment, the TDP-43 proteinopathy is a frontotemporal dementia (such as FTD-TDP-43 or other non-tau FTD).

TDP-43 proteinopathies, such as ALS, are known to be associated with mutations in the TDP-43 (TARDBP) gene in a subset of patients.

According to the Amyotrophic Lateral Sclerosis Online genetics Database (ALSoD; available at http://alsod.iop.kcl.ac.uk; see Abel et al., 2012, Hum. Mutat. 33:1345-1351), over fifty different mutations of the TARDBP gene have been identified in ALS patients (see Table 1 below, wherein the NCBI reference nucleotide sequence is NM_007375.3).

TABLE 1
TARDBP mutations associated with ALS

| Name | Code | Type | Original | Mutated | Intron/exon |
|---|---|---|---|---|---|
| 5′ UTR | c.-69C > T | Polymorphism | C | T | exon |
| 5′ UTR | c.-66G > T | Polymorphism | G | T | exon |
| p.Leu27 | c.81G > A | Polymorphism | G | A | intron |
| c.-12-54G > A | c.-12-54G > A | Polymorphism | G | A | intron |
| p.Ala66 | c.81G > A | Polymorphism | T | C | intron |
| p.Ser104 | c.312C > T | Polymorphism | G | A | intron |
| p.Lys137 | c.411A > G | Polymorphism | A | G | intron |
| c.403-80G > A | c.403-80G > A | Polymorphism | G | A | intron |
| c.543+112C > A | c.543+112C > A | Polymorphism | C | A | intron |
| Gly40Gly | G40G | Polymorphism | GGG | GGA | exon |
| Ala66Ala | A66A | Substitution | GCT | GCC | exon |
| Ala90Val | A90V | Substitution | GCT | GTT | exon |
| Asp169Gly | D169G | Substitution | GAT | GGT | exon |
| Asn267Ser | N267S | Substitution | AAT | AGT | exon |
| Gly287Ser | G287S | Substitution | GGT | AGT | exon |
| Gly290Ala | G290A | Substitution | GGT | GCT | exon |
| Ser292Asn | S292N | Substitution | AGC | AAC | exon |
| Gly294Ala | G294A | Substitution | GGG | GCG | exon |
| Gly294Val | G294V | Substitution | GGG | GTG | exon |
| Gly295Arg | G295R | Substitution | GGT | CGT | exon |
| Gly295Ser | G295S | Substitution | GGT | AGT | exon |
| Gly298Ser | G298S | Substitution | GGT | AGT | exon |
| Met311Val | M311V | Substitution | ATG | GTG | exon |
| Ala315Thr | A315T | Substitution | GCG | ACG | exon |
| Ala321Gly | A321G | Substitution | GCC | GGC | exon |
| Ala321Val | A321V | Substitution | GCC | GTC | exon |
| Gln331Lys | Q331K | Substitution | CAG | AAG | exon |
| Ser332Asn | S332N | Substitution | AGC | AAC | exon |
| Gly335Asp | G335D | Substitution | GGT | GAT | exon |
| Met337Val | M337V | Substitution | ATG | GTG | exon |
| Gln343Arg | Q343R | Substitution | CAG | CGG | exon |
| Asn345Lys | N345K | Substitution | AAC | AAA | exon |
| Gly348Cys | G348C | Substitution | GGC | TGC | exon |
| Gly348Val | G348V | Substitution | GGC | GTC | exon |
| Asn352Ser | N352S | Substitution | AAT | AGT | exon |
| Met359Val | M359V | Substitution | ATG | GTG | exon |
| Arg361Ser | R361S | Substitution | AGG | AGT | exon |
| Pro363Ala | P363A | Substitution | CCA | GCA | exon |
| Ala366Ala | A366A | Polymorphism | GCC | GCG | exon |
| Tyr374STOP | Y374X | Substitution | TAT | TAA | exon |
| Gly376Asp | G376D | Substitution | GGC | GAC | exon |
| Asn378Asp | N378D | Substitution | AAT | GAT | exon |
| Ser379Pro | S379P | Substitution | TCT | CCT | exon |
| Ser379Cys | S379C | Substitution | TCT | TGT | exon |
| Ala382Thr | A382T | Substitution | GCA | ACA | exon |
| Ala382Pro | A382P | Substitution | GCA | CCA | exon |
| Ile383Val | I383V | Substitution | ATT | GTT | exon |
| Ile383Thr | I383T | Substitution | ATT | ACT | exon |
| Gly384Arg | G384R | Substitution | GGT | CGT | exon |
| Trp385Gly | W385G | Substitution | TGG | GGG | exon |
| Asn390Asp | N390D | Substitution | AAT | GAT | exon |
| Asn390Ser | N390S | Substitution | AAT | AGT | exon |
| Ser393Leu | S393L | Substitution | TCG | TTG | exon |

An earlier study identified twenty-nine different missense mutations in the TARDBP gene with all but one (D169G) residing in the C-terminal glycine-rich domain encoded by exon 6 of TDP-43 (for example, see Pesiridis et al., 2009, Hum. Mol. Gen. 18 (review issue 2):R156-R162, the disclosures of which are incorporated by reference). In the study by Pesiridis et al., all missense mutations were observed in patients who were clinically diagnosed with a motor neuron disease, including eighteen fALS patients (n=1167 surveyed in all studies) and thirty sporadic sALS patients (n=2846 surveyed in all studies) that were not observed in control individuals (n=8117 surveyed in all studies). All TARDBP mutations exhibit an autosomal dominant pattern of inheritance and appear to have an equivalent gene dosage from both the wild-type (WT) and the mutant alleles. Mutations in the TDP-43 (TARDBP) gene have also been reported in patients with tau-negative frontotemporal dementia (see Benajiba et al., 2009, Ann. Neurol. 65:470-473, the disclosures of which are incorporated by reference).

Discovery of TARDBP mutations in both ALS and FTD patients reflects a recurring theme that these diseases are linked within a broad spectrum of neurodegenerative TDP-43 proteinopathies. Such mutations are thought to lead to disease pathology though a number of different mechanism, including:
(a) cytoplasmic mislocalisation of TDP-43;
(b) fragmentation of TDP-43;
(c) misfolding, aggregation, and insolubility of TDP-43; and/or
(d) phosphorylation and ubiquitination of TDP-43
   (see Scotter et. al, 2015, Neurotherapeutics 12(2): 352-363, the disclosures of which are incorporated by reference).

Thus, in one embodiment, the invention provides tacrolimus for use in the treatment of subject who has a mutation in the TDP-43 (TARDBP) gene. For example, the patient may carry a mutation in the TDP-43 (TARDBP) gene selected from those in Table 1 and/or identified in Pesiridis et al., 2009 (supra). Such patients include those with familial ALS and FTD.

However, it has also been found that abnormal TDP-43 expression levels and cytoplasmic accumulation may cause disease, leading to sporadic ALS, even if TDP-43 lacks a mutation. Studies have shown that levels and localisation of (non-mutated) TDP-43 may critically determine neurotoxicity (see Talbot, 2014, J Anat. 224(1):45-51; the disclosures of which are incorporated by reference).

Thus, in an alternative embodiment, the invention provides tacrolimus for use in the treatment of subject who does not have a mutation in the TDP-43 (TARDBP) gene but nevertheless exhibits an accumulation of TDP-43 in the central nervous system. For example, the treatment may be for a patient with sporadic ALS.

It will be appreciated by persons skilled in the art that the patient presenting with symptoms of a TDP-43 proteinopathy, such as ALS, may be tested to determine if they have a mutation in the TDP-43 (TARDBP) gene, prior to commencement of treatment with tacrolimus according to the invention. Suitable methods for carrying out such genotyping tests are well known in the art, for example in vitro hybridisation assays using oligonucleotide probes and/or PCR-based methods to amplify and sequence the TDP-43 (TARDBP) gene.

A key feature of the methods of the present invention is the unusually low dose of tacrolimus used to treat the patient with a TDP-43 proteinopathy. Specifically, tacrolimus is administered to the patient in a therapeutically-effective dose which does not cause immunosuppression in the subject. It will be appreciated by persons skilled in the art that such doses of tacrolimus may be administered by different routes, including but not limited to oral, topical, ocular, nasal, pulmonary, buccal, parenteral (intravenous, subcutaneous, and intramuscular), vaginal and rectal.

Without wishing to be bound by theory, it is believed that the mechanism by which tacrolimus provides a therapeutic effect in the present invention is not related to (that is, it is distinct from) the mechanism by which it achieves immunosuppression. Indeed, use at conventional immunosuppressant doses (i.e. at doses which achieve therapeutically effective levels of immunosuppression) is believed to be at best poorly effective in treating a patient with a TDP-43 proteinopathy. Tacrolimus has been found to have a therapeutic effect in the current invention at doses well below conventional immunosuppressant doses.

The term “does not cause immunosuppression” indicates that major side effects of immunosuppression do not occur in the patient. This results from a dose of tacrolimus being employed that does not substantially depress TNFα levels in the patient. It is believed that a dose of less than about 1.3 mg per day of tacrolimus in a 70 kg adult (pro-rata for other body weights) may be considered not to lead to immunosuppression.

However, because of individual personal variation the skilled person will be guided by the blood levels obtained (as indicated below).

In one embodiment, the tacrolimus is for administration (for example, orally) at a daily dose not exceeding 0.020 mg/kg, for example not exceeding 0.019 mg/kg, 0.018 mg/kg, 0.017 mg/kg, 0.016 mg/kg, 0.015 mg/kg, 0.014 mg/kg, 0.013 mg/kg, 0.012 mg/kg, 0.011 mg/kg, 0.010 mg/kg, 0.009 mg/kg, 0.008 mg/kg, 0.007 mg/kg, 0.006 mg/kg, 0.005 mg/kg, 0.004 mg/kg, 0.003 mg/kg, 0.002 mg/kg, or 0.001 mg/kg.

In a related embodiment, the tacrolimus is for administration (for example, orally) at a daily dose of at least 0.0005 mg/kg, for example at least 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.010 mg/kg, 0.011 mg/kg, 0.012 mg/kg, 0.013 mg/kg, 0.014 mg/kg, 0.015 mg/kg, 0.016 mg/kg, 0.017 mg/kg, 0.018 mg/kg, or 0.019 mg/kg.

It will be appreciated by persons skilled in the art that the dose of tacrolimus must be adequate to provide a therapeutic benefit to the patient. Although the precise dose required may differ from patient to patient, e.g. due to factors such as weight and metabolic rate, it is generally considered tacrolimus should be administered at a dose sufficient to produce a trough whole blood level of tacrolimus in the subject of at least 0.05 ng/ml. In one embodiment, tacrolimus may be administered at a dose sufficient to produce a trough whole blood level of tacrolimus in the subject of at least 0.075 ng/ml, for example at least 0.2 ng/ml or at least 0.3 ng/ml.

In a related embodiment, tacrolimus should be administered at a dose sufficient to produce a trough whole blood level of tacrolimus in the subject of less than 1.2 ng/ml, for example less than 1.1 ng/ml or less than 1.0 ng/ml.

Conveniently, tacrolimus is formulated for enteral administration, i.e. oral, gastric and/or rectal administration. For example, the tacrolimus may be formulated for oral administration, such as a tablet, capsule or other discrete unit dose.

Typically, tacrolimus is for daily administration, for example once, twice or three times a day (i.e. one dose only per day, two doses only per day, etc). However, therapeutically beneficial results may also be obtained by dosing at a frequency of less than once per day, for example once every 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days or more. Particularly suitable intervals for ease of patient use apart from daily may include on alternative days, once a week, once every 10 days, three times a month, once a fortnight or once a month.

A second aspect of the invention provides a unit dosage form comprising tacrolimus and a pharmaceutically-acceptable diluent, carrier or excipient, wherein the unit dosage form contains 0.05 mg to 1.3 mg of tacrolimus.

In one embodiment, the unit dosage form contains an amount of tacrolimus not exceeding 1.2 mg, for example not exceeding 0.75 mg, 0.6 mg or 0.4 mg of tacrolimus.

In a related embodiment, the unit dosage form contains an amount of tacrolimus equal to or exceeding 0.06 mg, for example equal to or exceeding 0.10 mg, or 0.15 mg of tacrolimus.

The tacrolimus may be provided as a solvate, such as a hydrate or alcoholate. For example, the tacrolimus may be employed as a hydrate such as the monohydrate (when weights are referred to herein they do not include the weight of the solvating molecule).

If desired an existing commercial product such as Prograf® may be purchased and its contents divided to produce the desired dose which may then be placed into a hard gelatin capsule for oral administration.

The unit dosage form may be liquid, for example a solution or suspension in a container, but it is considered preferable that the unit dose is non-liquid. Suitable solid unit dosage forms include tablets and capsules of which capsules are more apt.

Conveniently, the unit dosage form is suitable for oral administration, for example a tablet or capsule.

In some cases, the unit dosage form may comprise tacrolimus in suspension in a suitable vehicle. Non-limiting examples of vehicles for oral administration include phosphate-buffered saline (PBS), 5% dextrose in water (D5W) and a syrup. The unit dosage form may be formulated to stabilize the consistency of a dose over a period of storage and administration. In some cases, the unit dosage form may contain a solution of tacrolimus dissolved in a diluent such as water, saline, and buffers optionally containing an acceptable solubilizing agent. In favoured form, the composition comprises a solid dosage form. In some cases, the solid dosage form comprises a capsule, a caplet, a lozenge, a sachet, or a tablet. In some cases, the solid dosage form is a liquid-filled dosage form. In some cases, the solid dosage form is a solid-filled dosage form. In some cases, the solid dosage form is a solid-filled tablet, capsule, or caplet. In some cases, the solid-filled dosage form is a powder-filled dosage form. In some cases, the solid dosage form comprises a compound in the form of micronized particles, granules or microcapsulated agent. In some cases, the composition comprises an emulsion which may contain a surfactant. In some cases, the solid dosage form comprises one or more of lactose, sorbitol, maltitol, mannitol, cornstarch, potato starch, microcrystalline cellulose, hydroxypropyl cellulose, acacia, gelatin, colloidal silicon dioxide, croscarmellose sodium, talc, magnesium stearate, stearic acid, pharmaceutically-acceptable excipients, colorants, diluents, buffering agents, moistening agents, preservatives, flavoring agents, carriers, and binders. In some cases, the solid dosage form comprises one or more materials that facilitate manufacturing, processing or stability of the solid dosage form or a flavoring agent.

Examples of suitable tacrolimus formulations are disclosed in WO2005/020993, WO2005/020994, and WO2008/0145143 and WO2010/005980, the disclosures of which are incorporated herein by reference.

In one embodiment, the unit dosage form comprises a solid dispersion of tacrolimus in a dispersion medium comprising a vehicle and a stabilising compound (also referred to as stabilising agent).

It will be appreciated by persons skilled in the art that the unit dosage forms of the invention may be formulated for different routes of administration, including but not limited to oral, topical, ocular, nasal, pulmonary, buccal, parenteral (intravenous, subcutaneous, and intramuscular), vaginal and rectal. Additionally, administration from implants is possible. Suitable preparation forms include, for example, granules, powders, tablets, coated tablets, (micro) capsules, suppositories, syrups, emulsions, microemulsions (defined as optically isotropic thermodynamically stable systems consisting of water, oil and surfactant), liquid crystalline phases (defined as systems characterised by long-range order but short-range disorder; examples include lamellar, hexagonal and cubic phases, either water- or oil continuous), or their dispersed counterparts, gels, ointments, dispersions, suspensions, creams, aerosols, droplets or injectable solution in ampoule form and also preparations with protracted release of active compounds, in whose preparation excipients, diluents, adjuvants or carriers are customarily used.

Formulation strategies for drug delivery of tacrolimus are detailed in Patel et al., 2012, Int. J. Pharm. Investig. 2(4):169-175 (the disclosures of which are incorporated herein by reference).

In one embodiment, the pH in the unit dosage form is below 7 (e.g. as measured by re-dispersion of the composition in water), for example the pH may be in the range from 3.0 to 3.6. The pH may be provided by a stabilizing agent and/or be adjusted by an inorganic or organic acid or a mixture thereof.

Suitable stabilising compounds and stabilising agents for use in a composition of the invention include, but are not limited to, inorganic acids, inorganic bases, inorganic salts, organic acids, organic bases and pharmaceutically acceptable salts thereof.

The organic acid is preferably a mono-, di-, oligo or polycarboxylic acid. Non-limiting examples of useful organic acids are acetic acid, succinic acid, citric acid, tartaric acid, acrylic acid, benzoic acid, malic acid, maleic acid, oxalic acid and sorbic acid; and mixtures thereof. Preferred organic acids are selected from the group consisting of oxalic acid, tartaric acid and citric acid.

The pharmaceutically acceptable salt of an organic acid or inorganic acid is preferably an alkali metal salt or an alkaline earth metal salt. Preferred examples of such salts are sodium phosphate, sodium dihydrogen phosphate, disodium hydrogen phosphate, potassium phosphate, potassium dihydrogen phosphate, potassium hydrogen phosphate, calcium phosphate, dicalcium phosphate, sodium sulfate, potassium sulfate, calcium sulfate, sodium carbonate, sodium hydrogen carbonate, potassium carbonate, potassium hydrogen carbonate, calcium carbonate, magnesium carbonate, sodium acetate, potassium acetate, calcium acetate, sodium succinate, potassium succinate, calcium succinate, sodium citrate, potassium citrate, calcium citrate, sodium tartrate, potassium tartrate, calcium tartrate, zinc gluconate, and zinc sulphate.

Suitable inorganic salts include, but are not limited to, sodium chloride, potassium chloride, calcium chloride, and magnesium chloride.

Stabilised formulations of tacrolimus are described in WO 2011/100975, the disclosures of which are incorporated herein by reference.

A related, third aspect of the invention provides the use of tacrolimus in the preparation of a medicament for the treatment of a TDP-43 proteinopathy (such as ALS) in a human subject wherein the tacrolimus is for administration at a therapeutically-effective dose which does not cause immunosuppression in the subject.

A related, fourth aspect of the invention provides a method for treatment of a TDP-43 proteinopathy (such as ALS) in a human subject comprising administering tacrolimus at a therapeutically-effective dose which does not cause immunosuppression in the subject.

By “the treatment of a TDP-43 proteinopathy” we include the alleviation, in whole or in part, of the symptoms of the TDP-43 proteinopathy. For example, in the case of ALS, the treatment may result in a slowing or cessation of the decline in motor function in the patient The term “therapeutically effective” is construed accordingly.

Preferred embodiments of the third and fourth aspects of the invention are as described above in relation to the first and second aspects of the invention.

Preferences and options for a given aspect, feature or parameter of the invention should, unless the context indicates otherwise, be regarded as having been disclosed in combination with any and all preferences and options for all other aspects, features and parameters of the invention.

The listing or discussion of an apparently prior-published document in this specification should not necessarily be taken as an acknowledgement that the document is part of the state of the art or is common general knowledge.

Preferred, non-limiting examples which embody certain aspects of the invention will now be described, with reference to the following figures:

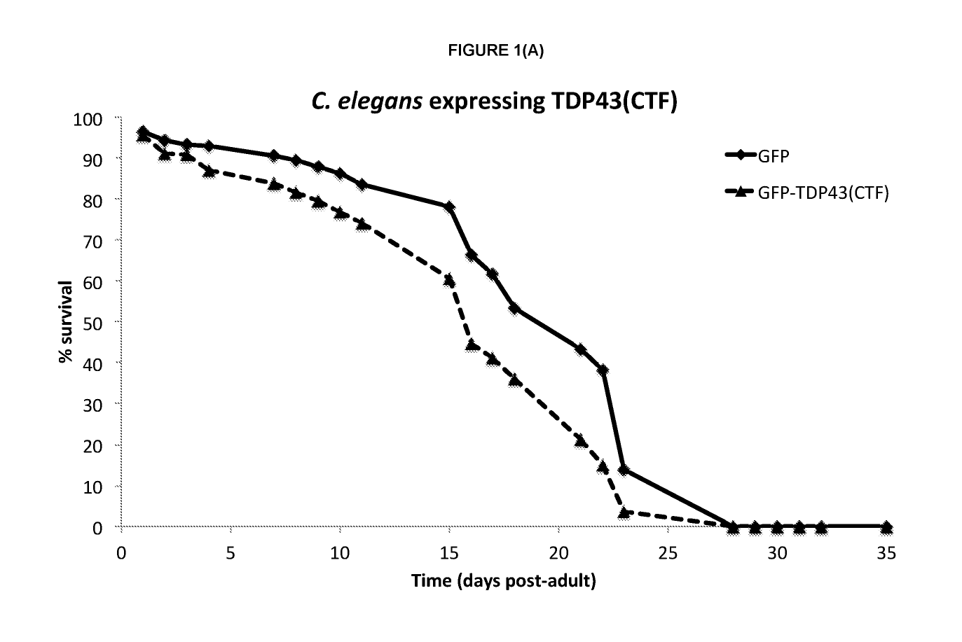

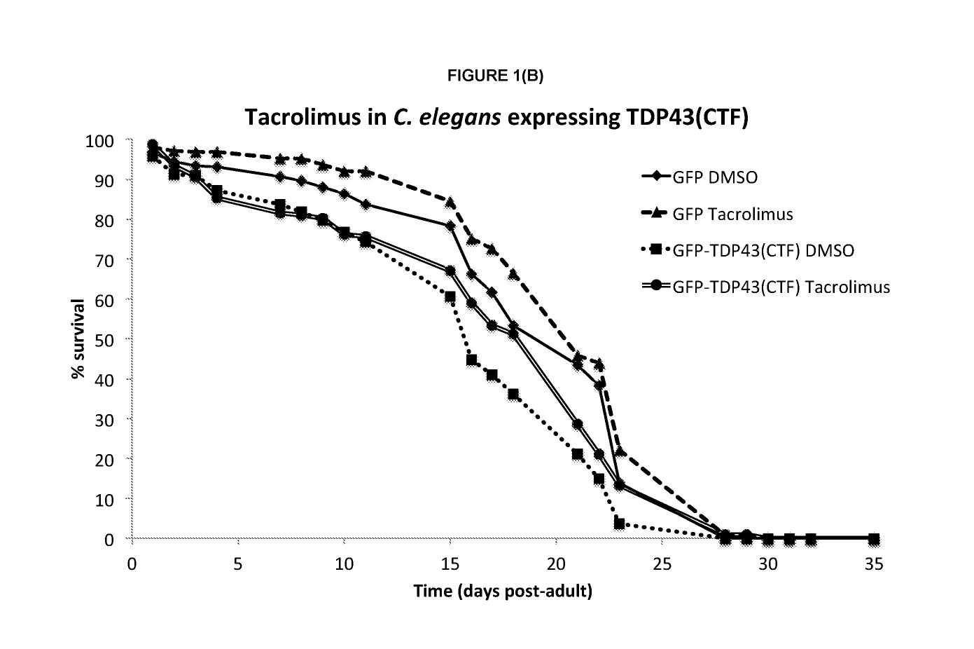

FIG. 1(A) shows the survival of C. elegans strains expressing GFP or GFP-TDP-43(CTF) in the body wall muscle. Expression of the C-terminal fragment of human TDP-43 causes a significant decrease in lifespan (p<0.005) relative to the control strain. (n=258 GFP, n=241 GFP-TDP-43(CTF))

FIG. 1(B) shows the survival of control and TDP-43-expressing C. elegans strains in the presence and absence of tacrolimus. Tacrolimus at a concentration of 10 μg/ml in the NGM agar causes a significant increase in lifespan in both control (GFP-expressing) worms and worms expressing the TDP-43 C-terminal fragment, relative to the vehicle-treated controls for each strain. (p<0.005 for GFP-TDP-43(CTF) DMSO vs. Tacrolimus, p<0.01 for GFP DMSO vs. Tacrolimus) (n=258 GFP DMSO, n=253 GFP Tacrolimus, n=241 GFP-TDP-43(CTF) DMSO, n=227 GFP-TDP-43(CTF) Tacrolimus)

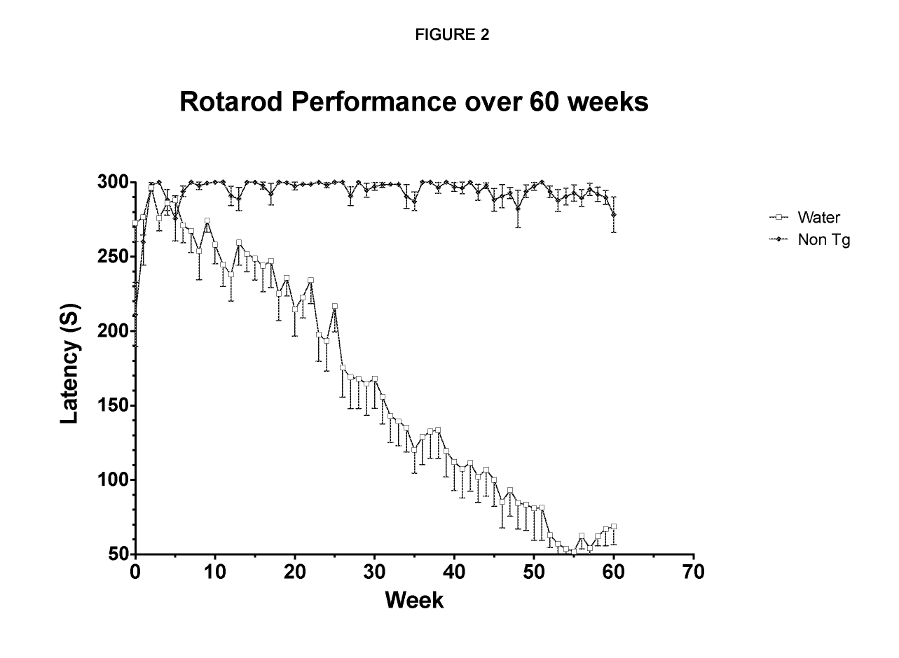

FIG. 2 shows the rotarod latency of TDP-43 (Q331K) mice (negative control group treated with water) compared with that of non-transgenic control mice (NTg) over 60 weeks (starting at 3 weeks of age). Mice expressing mutant (Q331K) human TDP-43 alone develop a mild but progressive decline in rotarod latency continuing throughout the course of the study. In contrast, the rotarod performance of the non-transgenic control mice remains steady. (n=15 TDP-43 (Q331K) water-treated control (“Water”) up to week 49 (thereafter numbers decline due to staggered breeding rounds), n=16 non-transgenic control (“Non Tg”) up to week 60)

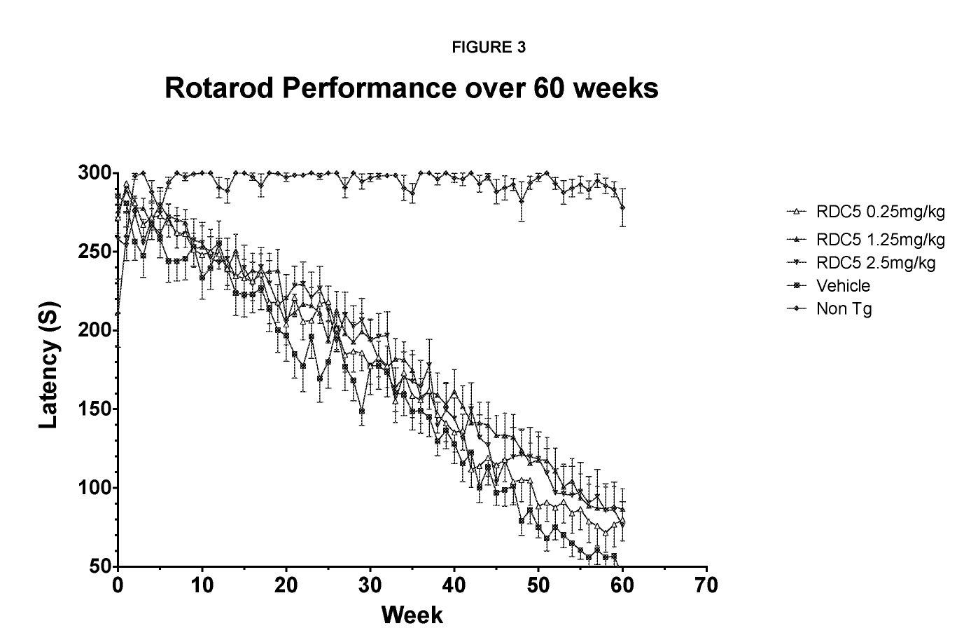

FIG. 3 shows the rotarod latency of TDP-43(Q331K) mice treated with Tacrolimus for 60 weeks (starting at 3 weeks of age) at 0.25 mg/kg per day (p.o.), 1.25 mg/kg per day (p.o.) or 2.5 mg/kg per day (p.o.) relative to a vehicle-treated control group. Tacrolimus treatment delays the progression of the decline in rotarod performance at all three doses tested. The effect is greater at the two higher doses. Interim statistical analysis (by two-way ANOVA with time and treatment as variables) indicates that this effect is significant. (n=21 vehicle, n=27 0.25 mg/kg tacrolimus, n=27 1.25 mg/kg tacrolimus, n=19 2.5 mg/kg tacrolimus, all up to 60 weeks)

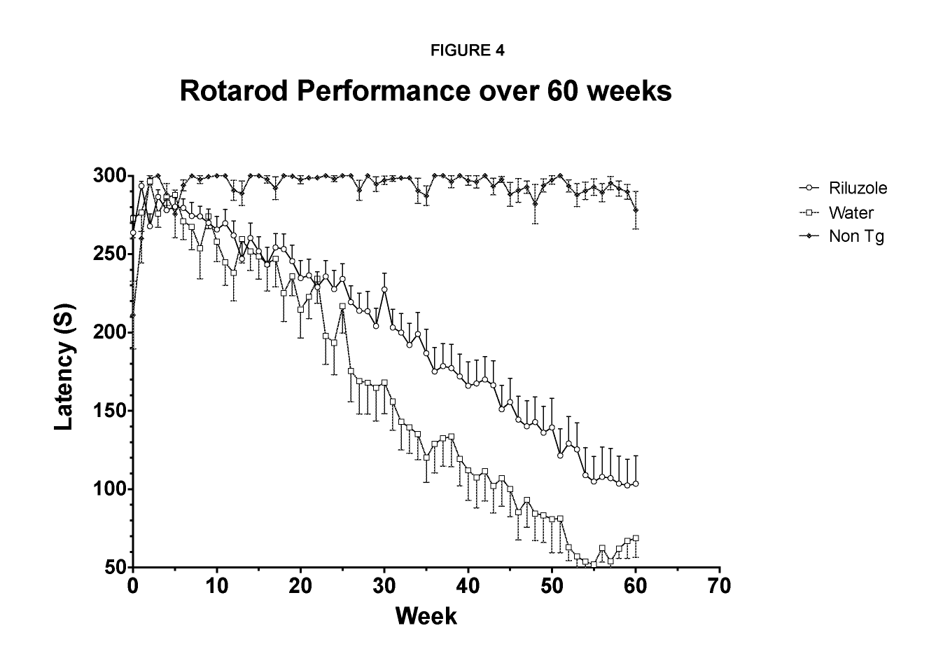

FIG. 4 shows the rotarod latency of TDP-43(Q331K) mice treated for up to 60 weeks with 10 mg/kg riluzole (p.o. per day) relative to a control group treated with water only. Treatment with 10 mg/kg riluzole delays the progression of the decline in rotarod performance. Interim statistical analysis (by two-way ANOVA with time and treatment as variables) indicates that the effect of riluzole is significant. (n=30 10 mg/kg riluzole up to week 49, n=15 water-treated control up to week 49 (thereafter numbers decline due to staggered breeding rounds))

### EXAMPLES

### Example 1—Effect of Tacrolimus in C. elegans Expressing TDP-43 C-Terminal Fragment

Materials and Methods

C. elegans Strains

C-terminal fragments of TDP-43 are associated with pathology in ALS and FTD patients. In particular, proteolytic cleavage at Arg208 has been reported to generate a pathogenic C-terminal fragment (see Wang et al., 2013, J. Biol. Chem. 288(13):9049-57 and Igaz et al., 2009, J. Biol. Chem. 284(13):8516-24; the disclosures of which are incorporated herein by reference). Therefore, a transgenic C. elegans strain was generated that expresses the C-terminal fragment (CTF) of human TDP-43 (aas 208-414) in the body wall muscle, fused via a short linker region to the C-terminus of a GFP tag. Expression is under the control of the myo-3 promoter.

A control C. elegans strain was generated in the same background, in which the GFP reporter only is expressed under the control of the myo-3 promoter.

These strains were crossed with worms carrying a mutation in the bus-5 gene, which confers drug-sensitivity, to produce drug-sensitive strains expressing GFP alone (“GFP”) or expressing the GFP-TDP-43 CTF fusion protein (“GFP-TDP-43(CTF)”).

C. elegans Lifespan Assays

C. elegans cultures were synchronised by incubating an asynchronous culture in basic sodium hypochlorite solution to kill everything but the bleach-resistant mature eggs. Approximately 300 eggs were added on to agar plates and then allowed to hatch and develop (to produce approximately 200 worms) in the presence of the test compound (or vehicle) and bacterial food source. When the worms reached the penultimate larval stage (L4; immediately prior to development of the next generation of mature eggs), approximately 150 worms were transferred to fresh growth plates containing 5-fluoro-2′-deoxyuridine (FUDR) and the test compound (or vehicle), along with the bacterial food source. FUDR was added to prevent further egg maturation and hatching to prevent interference from development of subsequent worm generations. Thereafter the worms were allowed to age in the presence of the test compound/vehicle and FUDR. From this point on, each ageing population was inspected daily and viability assessed by counting the number of dead worms.

The cumulative number of dead worms for each population was used to generate a survival/viability plot to determine chronological lifespan (CLS). Survival was analysed using the Kaplan-Meier log-rank survival analysis method, using the Online Application for Survival (OASIS; see http://sbi.postech.ac.kr/oasis). The principle of the analysis method is that the proportion of dead worms on each day is related to the size of the ‘at risk’ population (i.e. the number of remaining live worms), which declines as the number of worms in the study continues. By comparing the ‘observed’ number of dead worms in the treatment group against the ‘expected’ number of dead worms in the ‘at risk’ group (the treatment+control groups are combined as the best estimate of the ‘at risk’ group), it is possible to determine the significance of any differences between the ‘observed’ and ‘expected’ deaths using the Chi-Squared test. The cumulative Chi-Squared probability over all the days of the study then indicates whether there is a significant difference between the control and treatment groups (a Chi-Squared p-value of <0.05 is accepted as a significant effect). The predictive accuracy of the Kaplan-Meier method is dependent on having at least 100 subjects (worms) in the treatment and control groups. Approximately 150 worms were transferred to each duplicate assay plate to ensure this condition was satisfied. Furthermore, once individual assay plates had been compared, data from duplicate assay plates were combined to increase the predictive accuracy of the test.

Results and Conclusions

Expression of TDP-43 C-Terminal Fragment Decreases Lifespan in C. elegans

Survival curves for C. elegans strains expressing GFP only (“GFP”) or GFP fused to the N-terminus of the C-terminal fragment (aas 208-414) of human TDP-43 (“GFP-TDP-43(CTF)”), are shown in FIG. 1(A). Expression of the C-terminal fragment of human TDP-43 causes a significant reduction in lifespan, relative to expression of the GFP tag alone.

C-terminal fragments of TDP-43 are associated with pathology in ALS and FTD patients. The observation of a reduction in lifespan in worms expressing the pathogenic C-terminal fragment of TDP-43 is consistent with translation of the toxic effect to a nematode model, and provides a platform for the identification and testing of drugs that might ameliorate the toxicity of the C-terminal fragment

Tacrolimus Increases Lifespan in TDP-43-Expressing C. elegans

FIG. 1(B) shows survival curves for control (GFP-expressing) worms and worms expressing the C-terminal fragment of TDP-43 (GFP-TDP-43(CTF)) in the presence and absence of 10 μg/ml tacrolimus. At this concentration, tacrolimus has a significant effect on both strains, increasing the lifespan of both the control strain and the strain expressing the pathogenic TDP-43 fragment Therefore tacrolimus is able to ameliorate the lifespan-shortening effect of the TDP-43 C-terminal fragment, partially rescuing the lifespan deficit in these worms.

### Example 2—Effect of Tacrolimus in Mice Expressing WT and/or Q331K TDP-43

Materials and Methods

Mice

Transgenic mice expressing wild-type human TDP-43 or human TDP-43 carrying a point mutation (Q331K) have been developed and described by the Shaw lab (see Arnold et al., 2013, Proc. Natl. Acad. Sci. 110(8):E736-45 and Mitchell et al., 2015, Acta. Neuropathol. Commun. 3(1):36; the disclosures of which are incorporated herein by reference). The inserted constructs placed the cDNA for N-terminal myc-tagged wild-type or mutant TDP-43 under the control of the mouse prion promoter, resulting in expression in the CNS. Because the constructs do not contain the 3′UTR of the human TDP-43 gene, TDP-43 mRNA levels are not autoregulated and therefore TDP-43 levels in the transgenic strains reach 2-3 fold above endogenous levels.

Hemizygous lines for each construct were established (TDP-43(WT) and TDP-43(Q331K) respectively), and crossing these lines produced compound hemizygous animals (TDP-43(WTxQ331K)).

For this study, single transgenic mice were generated from existing mouse lines and genotyped (by PCR of DNA extracted from tail-tip samples) before reaching 3 weeks of age. Young breeding pairs (approx. 6-8 weeks old) of TDP-43(WT) and TDP-43(Q331K) were established to generate mice co-expressing both transgenes (TDP-43(WTxQ331K), together with single WT, Q331K and non-transgenic (NTg) littermates. Only the single TDP-43(Q331K) and double (TDP-43(WTxQ331K)) transgenic animals were required for this study. However, the double (TDP-43(WTxQ331K)) transgenic animals rapidly developed a disease phenotype (as previously described: see Mitchell et al., 2015, Acta. Neuropathol. Commun. 3(1):36) and did not survive long enough for dosing to commence. Therefore, only the single (TDP-43(Q331K)) transgenic animals were used to test the efficacy of tacrolimus or riluzole.

Multiple breeding rounds were required to achieve sufficient numbers of animals per treatment group and dosing/testing were staggered accordingly. All animals were tail-tipped for genotyping and ear-punched for identification at 2 weeks of age. Litter-mates were randomly distributed into each treatment group where possible (see below), with the aim of achieving equal numbers of males and females in each treatment group.

Because of the need to stagger dosing/testing, and the length of the study note that the current data set contains data for all animals only up to 49 weeks. From 50 weeks onwards, the n numbers in some groups decline as the animals from later breeding rounds continue to progress through the study.

Drug Treatment

Following a 2-day recovery period after tail-tipping, mice were acclimatised with a polypropylene oral gavage (delivering water only) each morning for 5 days. At 3 weeks of age (following the 5-day acclimatisation period) drug dosing commenced. Dosing was carried out 6 days a week by oral gavage with the following treatment groups:
Water, negative control
Vehicle (20% Cremophor RH40; 80% dehydrated ethanol (w/v)), negative control
Tacrolimus (2.5 mg/kg)
Tacrolimus (1.25 mg/kg)
Tacrolimus (0.25 mg/kg)
Riluzole (10 mg/kg in water), positive control

Dosing was continued for at least 60 weeks.

Based on pharmacokinetic analysis, a tacrolimus dose of 2 mg/kg/day in the mouse is believed to correspond to a human oral dose of about 0.33-0.44 mg/day oral for a 70 kg person.

Riluzole is currently the only FDA-approved drug for treatment of ALS and was therefore used as a positive control to confirm the validity of the model.

Behavioural and Phenotypic Assays

Mice were weighed after 3 days of oral gavage acclimatisation (2 days prior to the start of dosing), and thereafter on a weekly basis beginning on day 0 (the day before dosing commenced).

Mice were assessed by rotarod testing on a weekly basis. Each animal was acclimatised and trained on the rotarod 2 days prior to the start of dosing, by first undergoing a basic 2 minute acclimatisation at 5 rpm, and then undergoing 2×5 minute training sessions using a 2-20 rpm acceleration paradigm. On the following day (the day before dosing commenced, day 0), each animal was tested using a 5 minute 2-30 rpm paradigm to give a baseline reading. Thereafter, testing was conducted on a weekly basis (on day 7, 14, etc.) using a single 5 minute 2-30 rpm paradigm. Testing was conducted at the same time each afternoon.

General animal health and welfare was monitored throughout the dosing period and any unusual phenotypes or behaviours were recorded.

Results and Conclusions

Tacrolimus Delays the Decline in Motor Function in TDP-43(Q331K) Mice

Mice expressing only TDP-43(Q331K) develop a mild but progressive decline in motor function, along with abnormal hind limb splay and tremor. However this mutation does not appear to cause premature death, relative to non-transgenic or TDP-43(WT) animals (see Mitchell et al., supra). In this study this phenotype is manifested as a progressive decline in rotarod latency (FIG. 2). This decline occurs continuously from the start of the study, in contrast to the rotarod performance of the non-transgenic controls which remains steady up to at least one year of age (FIG. 2).

Treatment with tacrolimus delays the progression of motor decline at 0.25 mg/kg, 1.25 mg/kg and 2.5 mg/kg (FIG. 3). The effect is more marked at 1.25 mg/kg and 2.5 mg/kg than at 0.25 mg/kg. Interim statistical analysis (by two-way ANOVA with time and treatment as variables) indicates that this effect is significant.

Treatment with 10 mg/kg riluzole causes a similar delay in loss of motor function (FIG. 4). (Note that riluzole is administered in water rather than in the Cremophor/ethanol vehicle used for tacrolimus, so the appropriate control is a group treated with water only.) Therefore the only currently FDA-approved treatment for ALS is also effective in this model.

Previous mouse models expressing mutant TDP-43 have differed substantially in elements of their phenotype from the human disease pathology (see Perrin, 2014, Nature 507(7493):423-5 and Scotter et al., 2015, Neurotherapeutics 12(2):352-63; the disclosures of which are incorporated by reference). However the model used in the current study more faithfully reproduces key aspects of the human disease, including: the late-onset, age-related progressive decline in motor function; cytoplasmic accumulation of insoluble TDP-43 inclusions; motor neuron and cortical neuron loss, accompanied by micro- and astrogliosis; disorganisation of muscle fibres and degeneration of neuromuscular junctions (see Mitchell et al., supra). To our knowledge, this is the first study to show a significant effect of riluzole, the only licensed treatment for ALS, in a TDP-43 mouse model. This lends further support to the validity of this model, and indicates that the positive effect observed for tacrolimus can be extrapolated to the human disease situation.

### Example 3—Exemplary Unit Dosage Form of the Invention

A hard gelatine capsule was filled with the following composition:

| | Formulation | | Reference |
|---|---|---|---|
| Component | (%) | Function | Standard |
| Tacrolimus | 0.30 | Active ingredient | USP |
| Lactose monohydrate | 89.45 | Diluent | Ph Eur |
| Hydroxypropylmethyl | 6.00 | Binder | Ph Eur |
| cellulose | | | |
| Croscarmellose sodium | 4.00 | Super-disintegrant | Ph Eur |
| Magnesium stearate | 0.25 | Lubricant | Ph Eur |
| Ethanol | qs | Binder fluid | |

One capsule as above containing 0.3 mg tacrolimus was administered daily to healthy human volunteers for three successive days. No significant changes in blood TNF-α levels occurred as a result of the administration. Similarly, no change in TNF-α levels occurred as a result of administering 0.6 mg daily of tacrolimus to healthy volunteers. The average trough level of tacrolimus (level after 24 hours of administration of each dose) observed was approximately 220 pg/mL. The average peak level of tacrolimus observed was approximately 3700 pg/mL and the average area under the curve was approximately AUC O_t=23500 (h*pg/mL).

The above capsules may be used to provide the treatments described herein before.

Use of two such capsules simultaneously to provide a single dose of 0.6 mg would be expected to result in a trough level of about 440 pg/mL.

1. A method for treating a TDP-43 proteinopathy in a human subject in need thereof, comprising administering a therapeutically-effective dose of tacrolimus to the human subject, wherein the therapeutically-effective dose of tacrolimus does not cause immunosuppression in the subject.

2. The method of claim 1, wherein the TDP-43 proteinopathy is selected from the group consisting of amyotrophic lateral sclerosis (ALS), frontotemporal lobar dementias (such as FTD-TDP-43 and FTD-tau), other dementias (such as Alzheimer's disease and Lewy-body dementia), parkinsonism (including Parkinson's disease, Perry syndrome and ALS Parkinsonism-dementia complex of Guam), polyglutamine diseases (such as Huntington's disease) and myopathies (such as sporadic inclusion body myositis).

3. The method of claim 2, wherein the TDP-43 proteinopathy is amyotrophic lateral sclerosis (ALS).

4. The method of claim 2, wherein the subject has a mutation in the TDP-43 (TARDBP) gene.

5. The method of claim 2, wherein the subject does not have a mutation in the TDP-43 (TARDBP) gene

6. The method of claim 1, wherein the tacrolimus is administered a daily dose not exceeding 0.020 mg/kg.

7. The method of claim 6, wherein the tacrolimus is administered at a daily dose of at least 0.0005 mg/kg, for example.

8. The method of claim 1, wherein the tacrolimus is administered at a dose sufficient to produce a trough whole blood level of tacrolimus in the subject of at least 0.05 ng/ml.

9. The method of claim 8, wherein the trough whole blood level is at least 0.075 ng/ml.

10. The method of claim 9, wherein the trough whole blood level is less than 1.2 ng/ml.

11. The method of claim 1, wherein the tacrolimus is administered by enteral administration, and optionally wherein the tacrolimus is administered by oral, gastric or rectal administration.

12. The method of claim 11, wherein the tacrolimus is administered orally, and optionally, wherein the tacrolimus is administered in the form of a tablet or capsule.

13. The method of claim 1, wherein the tacrolimus is administered daily.

14. A unit dosage form comprising tacrolimus and a pharmaceutically-acceptable diluent, carrier or excipient, wherein the unit dosage form comprises 0.05 mg to 1.3 mg of tacrolimus.

15. The unit dosage form of claim 14, wherein the unit dosage form comprises an amount of tacrolimus not exceeding 1.2 mg.

16. The unit dosage form of claim 14 wherein the unit dosage form comprises an amount of tacrolimus equal to or exceeding 0.06 mg.

17. The unit dosage form of claim 14 suitable for oral administration, and optionally is a tablet or capsule.

18. Use of tacrolimus in the preparation of a medicament for the treatment of a TDP-43 proteinopathy in a human subject wherein the tacrolimus is for administration at a therapeutically-effective dose which does not cause immunosuppression in the subject.

19. The use of tacrolimus in the preparation of a medicament according to claim 18 wherein the TDP-43 proteinopathy is amyotrophic lateral sclerosis (ALS).

20. (canceled)

21. (canceled)

22. The method of claim 6, wherein the tacrolimus daily dose does not exceed 0.019 mg/kg, 0.018 mg/kg, 0.017 mg/kg, 0.016 mg/kg, 0.015 mg/kg, 0.014 mg/kg, 0.013 mg/kg, 0.012 mg/kg, 0.011 mg/kg, 0.010 mg/kg, 0.009 mg/kg, 0.008 mg/kg, 0.007 mg/kg, 0.006 mg/kg, 0.005 mg/kg, 0.004 mg/kg, 0.003 mg/kg, 0.002 mg/kg, or 0.001 mg/kg.

23. The method of claim 7, wherein the tacrolimus is administered at a daily dose of at least 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.010 mg/kg, 0.011 mg/kg, 0.012 mg/kg, 0.013 mg/kg, 0.014 mg/kg, 0.015 mg/kg, 0.016 mg/kg, 0.017 mg/kg, 0.018 mg/kg, or 0.019 mg/kg.

24. The method of claim 9, wherein the trough whole blood level is at least 0.2 ng/ml or at least 0.3 ng/ml.

25. The method of claim 10, wherein the trough whole blood level is less than 1.1 ng/ml or less than 1.0 ng/ml.

26. The unit dosage form of claim 15, wherein the unit dosage form comprises an amount of tacrolimus not exceeding 0.75 mg, 0.6 mg or 0.4 mg of tacrolimus.

27. The unit dosage form of claim 16, wherein the unit dosage form comprises an amount of tacrolimus equal to or exceeding 0.10 mg, or 0.15 mg of tacrolimus
